(19) United States
(12) Patent Application Publication
(10) Pub. No.: US 2014/0093910 A1
(43) Pub. Date: Apr. 3, 2014

(54) D5 DESATURASE-DEFECTIVE MUTANT GENE AND USE THEREOF

(71) Applicants: Inna Khozin-Goldberg, Midreshet Ben-Gurion (IL); Zvi Hacohen, Omer (IL); Sammy Boussiba, Omer (IL); Avigad Vonshak, Midreshet Ben-Gurion (IL); Umidjon Iskandrov, Sede Boker Campus (IL)

(72) Inventors: Inna Khozin-Goldberg, Midreshet Ben-Gurion (IL); Zvi Hacohen, Omer (IL); Sammy Boussiba, Omer (IL); Avigad Vonshak, Midreshet Ben-Gurion (IL); Umidjon Iskandrov, Sede Boker Campus (IL)

(73) Assignee: Bengurion University of the Negev Research and Development Authority, Beer-Sheva (IL)

(21) Appl. No.: 13/703,100

(22) PCT Filed: Jun. 9, 2011
(86) PCT No.: PCT/IL11/00451

§ 371 Date: Jun. 19, 2013

Related U.S. Application Data
(60) Provisional application No. 61/353,221, filed on Jun. 10, 2010.

(51) Int. Cl. C12N 9/00; C12N 15/79

(52) U.S. Cl. CPC C12N 9/0071; C12N 15/79
USPC 435/32; 536/23.6; 435/320.1; 435/257.2; 435/471

(57) ABSTRACT

It is an object of the present invention to provide a delta-5 desaturase-defective gene and uses of the gene and/or the mutant in algal transformation.

### FIELD OF THE INVENTION

The present invention relates to isolated nucleic acid sequences of a Δ5 desaturase-defective gene of the micro-alga Parietochloris incisa and to the use of a mutant containing such nucleic acids.

### BACKGROUND OF THE INVENTION

Dihomo-γ-linolenic acid (DGLA) (also known as 8,11,14-eicosatrienoic acid) is an industrially-important fatty acid that can be used for pharmaceutical and nutritional applications, in feed for aquaculture and animals. Studies in mammals have shown that DGLA competes with arachidonic acid (ARA, 20:4^{Δ5,8,11,14}) in binding to cyclooxygenase, and thus causes a reduction in the levels of the pro-inflammatory dienoic eicosanoids, PGE₂ and LP₄, which are derived from ARA, and an increase in prostaglandin PGE₁. The latter, which is derived from DGLA, has been shown to have a positive effect in a variety of diseases, e.g., atopic eczema, psoriasis, asthma and arthritis, due to its anti-inflammatory properties and modulation of vascular reactivity.

DGLA is, therefore, of potential pharmacological significance. However, the lack of sources for large scale production has prevented its clinical research and, consequently, its neutriceutical or pharmaceutical use. Whereas higher plants or fungi and algae accumulate polyunsaturated fatty acids (PUFA), DGLA normally occurs only as an intermediate in the biosynthesis of ARA; it is not appreciably accumulated in any organism. Instead, GLA-rich oil from several plant species is utilized as a DGLA precursor. However, the conversion of GLA to DGLA in the body is, under certain conditions, e.g., low calcium, significantly diminished, and in such cases, GLA cannot replace DGLA

As mentioned above, DGLA serves as an intermediate in the biosynthesis of ARA, the conversion of DGLA to ARA being mediated by the enzyme Δ5 desaturase.

Until recently, the only known source of DGLA was a Δ5 desaturase-deficient mutant of the fungus Mortierella alpina. However, PUFAs produced by this fungal mutant have an unfavorably low DGLA/ARA ratio. A further disadvantage of the fungal-derived PUFAs is that they are susceptible to oxidation and synthetic antioxidants need to be added to prevent deterioration by oxidation. Since the oxidation is a chain reaction, even a small amount of oxygen can destroy PUFA rapidly.

Plant oils are capable of producing various PUFAs. However, those PUFAs produced by higher plants are restricted to chains of up to 18 carbon atoms. Microalgae, on the other hand, are known to produce PUFAs of up to 22 carbon atoms long. Further, PUFA-containing oil derived from algae contains endogenous antioxidant—β-carotene.

The freshwater alga Parietochloris incisa is the richest plant source of the PUFA ARA.

Algae biotechnology is currently used in the production of, for example, food additives, cosmetics, animal feed additives, pigments, polysaccharides, fatty acids and biomass. Progress in algal transgenics promises a much broader field of application; molecular farming. However, transgenesis in algae is a complex, albeit fast growing, technology. Currently genetic tools for algal transformation, such as selectable marker genes, are scarce and only a few algae species are accessible to genetic transformation.

In order to develop a reliable transformation system for green algae, various approaches are used, including sets of selection markers (antibiotic, herbicide resistance) and reporter genes (mainly, foreign genes encoding GFP or GUS). The main obstacles in genetic transformation of green algae are poor expression of foreign genes and poor penetration of foreign DNA through the tough cell wall.

Large scale cultivation of microalgae suffers from problems of contamination by various environmental stresses such as faster growing species in open ponds and photoinhibition by high light intensities. Genetic modification of microalgae may be used to introduce new useful traits such as herbicide resistance, tolerance to high light intensity, tolerance to high salinity caused by water evaporation, etc. Moreover, genetic modification of microalgae may aid in the metabolic engineering of algae to produce various nutritionally and pharmaceutically important PUFA.

WO 2009/022323 (to Cohen et al.) describes a process for producing DGLA from a mutant strain of the micro-alga Parietochloris incisa that is defective in its Δ5 desaturase (Δ5D) gene, and a process for recovering DGLA-containing lipids therefrom.

However, the nucleic acid sequence coding for the defective Δ5D enzyme is not disclosed nor is the mutation site identified.

### SUMMARY OF THE INVENTION

It is an object of the present invention to provide a gene that encodes a defective form of the Δ5 desaturase gene. The Δ5 desaturase-defective gene produces a biochemically inactive peptide interfering with the conversion of DGLA to ARA, rendering an algae mutant carrying the defective gene, DGLA rich.

Another object of the present invention is to provide a selectable marker (e.g., reporter gene) for algal genetic transformation, which is advantageously an endogenous algal gene rather than a foreign gene. For example, the present invention provides use of a Δ5 desaturase-defective gene as a selective marker for algal transformation. According to another embodiment functional complementation of the Δ5 desaturase mutant with the wild type Δ5 desaturase cDNA is used to select for transformed algae.

### BRIEF DESCRIPTION OF THE FIGURES

The invention will now be described in relation to certain examples and preferred embodiments with reference to the following illustrative figures so that it may be more fully understood. In the drawings:

FIG. 1 shows a fragment of the MutPiDes5 cDNA and its deduced amino acid sequence including the mutation site, according to an embodiment of the invention;

FIG. 2 shows a PCR amplified fragment of the MutPiDes5 genomic sequence containing the mutation site, according to one embodiment of the invention;

FIG. 3 shows a GS-MS spectrum of the peak corresponding to DGLA pyrrolidine derivative;

FIG. 4 shows a comparison of partial cDNAs of WT (WtPiDes5) and Mutant (MutPiDes5) P. incisa Δ5 desaturase genes, according to one embodiment of the invention; and

FIG. 5 shows the time-course of VLC-PUFA biosynthesis gene expression in WT and mutant P. incisa under N-starvation (Time 0—log phase culture), according to an embodiment of the invention.

### DETAILED DESCRIPTION OF THE INVENTION

According to one embodiment of the invention a Δ5 desaturase-deficient algal strain rich in dihomo-γ-linolenic acid (DGLA, 20:3^{Δ8,11,14}) was isolated. The defective Δ5 desaturase gene was sequenced and the mutation site was identified.

Functionally, the natural Δ5 desaturase gene (WTPiD5DES) (Gen Bank accession number GU390533) represents a protein having a molecular weight of approximately 119.65 kDa (based on: http://www.encorbio.com/protocols/Prot-MW.htm). It is involved in the synthesis of highly unsaturated fatty acids such as arachidonic acid (ARA).

The mutated PiD5DES gene (MutPiD5DES) (SEQ ID NO. 1) produces a severely truncated peptide which affects the transcriptional up-regulation of all genes involved in long chain polyunsaturated fatty acids (LC-PUFA) biosynthesis, severely decreasing transcription of these genes and enabling increased accumulation of oleic acid and DGLA in the mutant.

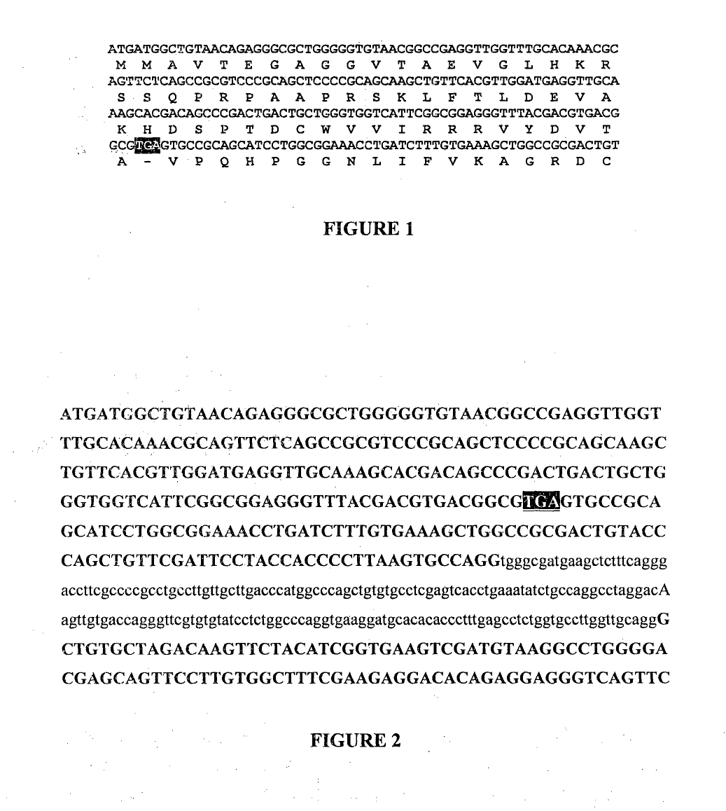

Structurally, the nucleotide sequence of MutPiD5DES (see, e.g. SEQ ID NO:1, isolated from mutated P. incisa) encodes an ORF of 1446 by nucleotides encoding 482 residues of the mutant Δ5 desaturase gene. FIG. 1 shows a fragment of MutPiDes5 cDNA and its deduced amino acid sequence including the mutation site (highlighted).

A 570 by nucleotide sequence starting from the start codon ATG and containing the mutation site and a 192 by intron was PCR amplified from genomic DNA. Shown in FIG. 2 is the PCR amplified fragment of the MutPiDes5 genomic sequence containing the mutation site (highlighted), a single point mutation in a tryptophan (W) encoding codon, upstream of the HPGG quartet (that is highly conserved within a fused cytochrome b5 domain in all cloned Δ5 and Δ6 desaturases regardless of their origin). In FIG. 2 the lower case letters represent the intron.

The mutation is stable as it did not revert during 3 years of sub-culturing.

Methods of detecting MutPiD5DES nucleic acids and expression of MutPiD5DES may be useful for confirming transgenesis, for example, algal transgenesis.

According to one embodiment of the invention there is provided an isolated nucleic acid molecule comprising at least a 186 by portion of the nucleotide sequence of SEQ ID NO. 1, said portion comprising the start codon ATG and the mutation site at bp 186.

According to some embodiments the molecule may contain the full length of SEQ ID NO. 1. The nucleic acid molecule may be cDNA or genomic DNA molecule.

According to another embodiment of the invention there is provided a vector comprising the isolated nucleic acid molecule.

According to yet another embodiment of the invention there is provided an isolated fresh water green algal cell comprising the nucleic acid molecule. According to some embodiments the alga is Parietochloris incisa or a close species.

According to another embodiment of the invention there is provided a vector for algal transformation, the vector comprising a plant derived promoter (such as 35S, RBSC, etc.), a WT PiD5DES gene and a gene to select for stable transformants (for example, a gene for herbicide or antibiotic resistance).

Another embodiment of the invention provides a method for transformation of algae, the method comprising: introducing into a MutPiDes5 mutant a vector as described above; selecting stable transformants (for example, based on resistance to herbicides); and analyzing the FA composition of the MutPiDes5 mutant for the emergence of ARA.

Some examples will now be described to further illustrate the invention and to demonstrate how embodiments of the invention may be carried-out in practice. In the Examples the isolation and use of a PiD5Des gene and mutant from Parietochloris incisa is described, however other algae may be used. These examples are intended only to exemplify the invention and not to limit the scope of the invention.

### EXAMPLES

1) Isolation of PiD5DES Mutant (p127)

Parietochloris incisa (Trebouxiophyceae, Chlorophyta), classified by Watanabe et al. (Parietochloris incisa comb. nov. (Trebouxiophyceae, Chlorophyta), Phycol. Res. 44 (1996) 107-108), was isolated from a snow water sample from Mt. Tateyama (Japan).

### 1a) Mutagenesis

During cell division, P. incisa produces cell aggregates. To isolate single cells, aliquots of log-phase culture were sonicated in water bath and observed by a light microscope (Zeiss). Ten mL of suspension, containing mostly single cells, were exposed to the mutagen, 1-methyl-3-nitro-nitrosoguanidine (MNNG, Sigma-Aldrich, St. Louis, Mo.) at a final concentration of 100 μg/mL for 1 h in an incubator shaker. The stock solution of MNNG (5 mg/mL) was prepared in dimethyl sulfoxide (DMSO) to ease the penetration of the mutagen across the tough cell wall of the alga. The cells were pelleted and washed several times with BG-11 medium. Finally, the cultures were sonicated in 10 mL of fresh medium, and cell numbers of untreated and treated cultures were counted. The cultures were sequentially diluted to 1000 cells per mL and plated on BG-11 agar plates. Plates were maintained under fluorescent light at room (25° C.) and low (15° C.) temperature. Colonies, which showed decreased performance (as estimated by decreased pigmentation and poor growth relative to the wild type) at low temperature, were selected and grown in liquid medium.

### 1b) Growth Conditions

Cultures were cultivated on BG-11 nutrient medium in 1 L glass columns under controlled temperature and light conditions. The columns were placed in a temperature regulated water bath at 25° C. and 15° C. and illuminated by cool white fluorescent lights from one side at a light intensity of 170 μmol photon m⁻²s⁻¹. Light intensity was measured at the middle and the center of the empty column with a quantum meter (Lamda L1-185, LiCOR, USA). The cultures were provided with a continuous bubbling of air and CO₂ mixture (98.5:1.5, v/v) from the bottom of the column. For nitrogen-starvation experiments, NaNO₃ was omitted from the medium and ferric ammonium citrate was substituted by ferric citrate.

Growth of the cultures was estimated on the basis of chlorophyll volumetric content and dry weight measurements. Chlorophyll's content (μg/mL) was measured in DMSO extracts, The biomass concentration was estimated by dry weight determination on pre-weighed glass fiber paper filters (Schleicher & Schuell Co.).

### 2) Lipid Analysis

### 2a) Fatty Acid Analysis

Fatty acid profile and content in the samples were determined as their methyl esters by capillary GC. Transmethylation of fatty acids were carried out by incubation of the freeze-dried cells, total lipid extracts, or individual lipids, in dry methanol containing 2% H₂SO₄ (v/v) at 70° C. for 1.5 h under argon atmosphere and continuous mixing. Heptadecanoic acid (Sigma-Aldrich, St. Louis, Mo.) was added as an internal standard.

Gas chromatographic analysis of FAMEs was performed on a ZB-WAX+(Phenomenex, USA) fused silica capillary column (30×0.32 mm) on Trace GC ultra Gas Chromatograph (Thermo, Italy) equipped with a flame ionization detector (FID) and a programmed temperature vaporizing (PTV) injector. The FID temperature was fixed at 280° C.; and a PTV injector was programmed to increase the temperature from 40° C. at time of injection to 300° C. at time of sample transfer. The oven temperature was programmed as follows: initial temperature of 130° C. was maintained for 1 min, then raised to 200° C. at a rate of 10° C. min⁻¹ and hold for 6 min, then raised to 230° C. at a rate of 15° C. min⁻¹ for 2 min. Helium was used as a carrier gas. FAMEs were identified by co-chromatography with authentic standards (Sigma-Aldrich) and by GC-MS (HP 5890 equipped with a mass selective detector HP 5971A.) as their pyrrolidine derivatives utilizing HP-5 capillary column (Aglient, USA) with a liner temperature gradient from 120 to 300° C. Pyrrolidide derivatives were prepared by reacting FAME with pyrrolidine in the presence of acetic acid.

3) Obtaining and characterizing the PiD5DES mutant sequence

### 3a) DNA and RNA Manipulation

RNA was isolated from cells harvested from log phase cultures (Time 0) and cells were cultured on nitrogen-free medium for 1.5, 3, 7 and 14 d according to Iskandarov et al. (Lipids 44 (2009) 545-554), and Iskandarov, U., Khozin-Goldberg, I. and Cohen, Z. (2010) Identification and characterization of Δ12, Δ6, and Δ5 desaturases from the green microalga Parietochloris incisa. Lipids (in press):DOI 10.1007/s11745-010-3421-4.

Genomic DNA of P. incisa was isolated as described by Doyle and Doyle (Phytochem. Bull. 19 (1987) 11-15) with minor modifications.

An open-reading frame (ORF) of Δ5 desaturase was PCR-amplified from cDNA with a proof-reading PfuUltra II fusion HS DNA polymerase (Stratagene, La Jolla, Calif.), cloned to E. coli through pGEM T-Easy vector (Promega, Madison, Wis.) and sequenced (ABI PRISM 3100 Genetic Analyzer). A fragment of the Δ5 desaturase gene corresponding to the mutation site in genomic DNA was amplified by PCR with PfuUltra II fusion HS DNA polymerase using the gene specific primers Des5For (5′-CCAAAGCTTAAAATGATGGCTGTAACAGA-3′) and Des5Rev (5′-TGTACGCCAAGTCGCTGACCATCC-3′), on DNA isolated from mutant P. incisa cells.

### 3b) Functional Characterization of the MutPiDes5 Gene

The ORF of 1446 by nucleotides encoding 482 residues of the mutant Δ5 desaturase gene was cloned into pYES2 (Invitrogen, Carlsbad, Calif., USA), yielding the pYMutPiDes5 construct. Saccharomyces cerevisiae (strain W303) was transformed with the construct as known in the art.

### 3c) RNA Isolation

Aliquots of the cultures were filtered through a glass fiber filter (GF-52, Schleicher & Schuell, Germany); cells were collected by scraping and immediately flash-frozen in liquid nitrogen and stored at −80° C. for further use. Total RNA was isolated by procedures known in the art. Three independent RNA isolations were conducted for each time point. The total RNA samples were treated with RNAase-free Baseline-ZERO™ DNAase (Epicentre Technologies, Madison, Wis., USA) before being used in cDNA synthesis for real-time PCR experiments.

3d) Gene cloning of partial sequences of the wild type Δ5 desaturase and actin genes were obtained by PCR (ReddyMix PCR Master Mix, Thermo Scientific, Surrey, UK) using the degenerate primers Des5For- ATH RAI GRI AAR GTI TAY GAY GT; Des5 Rev -: GGI AYI KWI TSD ATR TCI GGR TC; non-degenerate ActF-AGA TCT GGC ACC ACA CCT TCT TCA; and ActR-TGT TGT TGT AGA GGT CCT TGC GGA). To generate the full-length cDNAs, 3′-and 5′-rapid amplification of the cDNA ends (RACE) was performed using a BD Smart™ RACE cDNA Amplification Kit (BD Biosciences Clontech, Foster City, Calif., USA). Gene specific primers were designed and RACE PCR reactions were conducted using 5′ and 3′-RACE-Ready cDNAs made from 1 μg total RNA of N-starved cells with 50x BD Advantage 2 polymerase mix (Clontech Laboratories Inc., Mountain View, Calif., USA). The PCR products of the expected sizes were excised, purified from the gel (NucleoSpin Extract II purification kit, Machery-Nagel, Duren, Germany) and ligated into a pGEM T-Easy vector (Promega, Madison, Wis., USA). The full-length cDNAs were assembled based on the sequences of the 5′ and 3′ RACE fragments.

### 3e) Semi Quantitative PCR

The cDNA samples for semi quantitative PCR were synthesized using 1 μg of Dnase treated total RNA in a total volume of 20-μL, using random hexamer (Verso™ cDNA Kit, ABgene, UK). Each 20-μL cDNA reaction mixture was then 7-fold and 10-fold diluted with PCR grade water to amplify the fragments of the actin and LC-PUFA biosynthesis genes, respectively. This was done due to the substantially higher expression of desaturases in the WT. PCR products were visualized in 2% agarose gel.

3f) translation of cDNA

An amino acid sequence was deduced for the MutPiDes5 cDNA. The translation was done using the Translate tool program (Expasy Proteomics, http://www.expasy.ch/tools/dna.html).

### Results

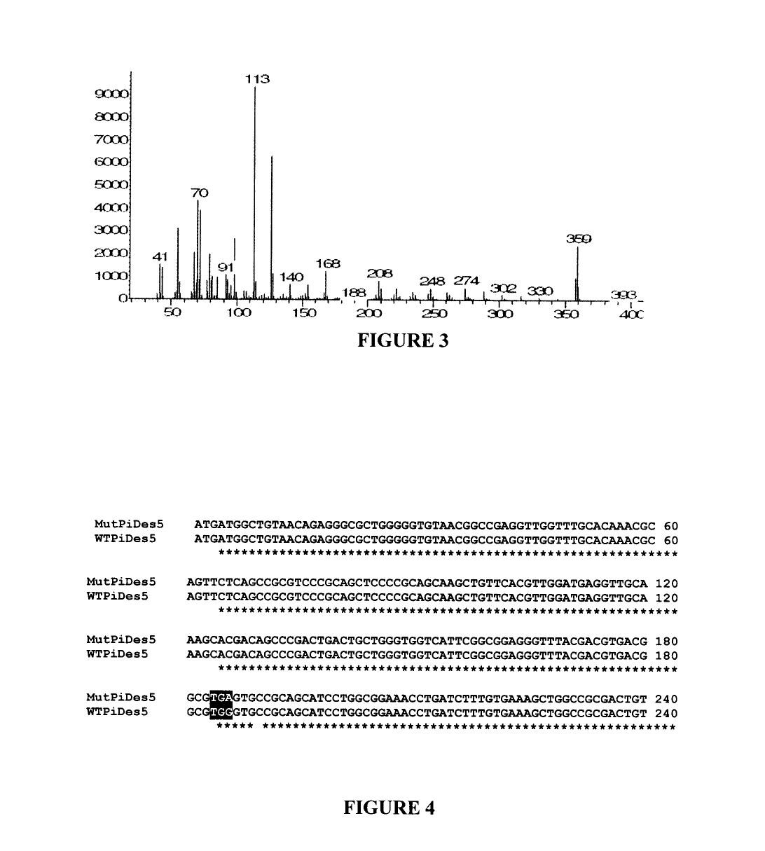

Cultures of P. incisa were chemically mutagenized with N-methyl-N′-nitro-N-nitrosoguanidine, as described above. Several colonies with reduced growth at low temperature (15 ° C.) were isolated and analyzed for fatty acid (FA) composition, as described above. Following growth on liquid medium, one of the colonies (P127), proved to be deficient in ARA; instead, its precursor, DGLA was accumulated. The chemical structure of DGLA was confirmed by GC-MS of the pyrollidine derivatives of the FA (FIG. 3). Further, FA composition and content of the WT and P127 strains were compared at the log-phase growth and after 14 days of nitrogen starvation, triggering TAG accumulation.

ARA was detected in the mutant at very low levels (less than 0.2% TFA) in comparison to over 20 and 58% in the wild type, after 2 days cultivation on nitrogen replete and 14 day nitrogen starvation, respectively (Table 1).

TABLE 1
Fatty acid composition (relative percentage w/w) and content of the wild type
and Δ5 desaturase mutant (P127) after 2 days cultivation on nitrogen
replete (+N) and after 14 days on nitrogen deplete (−N) media.

| | Culture | Fatty acid composition (% of total) | | | | | | | |
|---|---|---|---|---|---|---|---|---|---|
| strain | conditions | 16:0 | 16:1 | 16:1 | 16:2-ω6 | 16:3-ω3 | 18:0 | 18:1-ω9 | 18:1-ω7 |
| WT | +N | 17.0 | 2.8 | 1.1 | 3.7 | 3.4 | 4.1 | 9.7 | 1.9 |
| P127 | | 16.6 | 2.4 | 1.1 | 2.9 | 4.6 | 2.8 | 14.3 | 2.3 |
| WT | −N | 9.3 | 0.3 | 0.2 | 0.2 | 0.3 | 3.1 | 10.8 | 4.0 |
| P127 | | 7.7 | 0.2 | 0.2 | 0.3 | 0.2 | 2.0 | 36.4 | 3.0 |

| | | Fatty acid composition (% of total) | | | | | | | | |
|---|---|---|---|---|---|---|---|---|---|---|
| | Culture | | | | 20:3-ω6 | 20:4-ω6 | | 20:5-ω3 | TFA | DW |
| strain | conditions | 18:2-ω6 | 18:3-ω6 | 18:3-ω3 | DGLA | ARA | 20:4-ω3 | EPA | (% DW) | g/l |
| WT | +N | 19.8 | 2.4 | 7.9 | 0.7 | 22.5 | — | 1.1 | 10.0 | 1.9 |
| P127 | | 19.2 | 2.8 | 11.8 | 16.7 | trace | 1.0 | — | 9.5 | 2.0 |
| WT | −N | 9.0 | 0.9 | 0.7 | 1.1 | 57.7 | — | 0.8 | 33.6 | 5.1 |
| P127 | | 13.9 | 1.1 | 1.1 | 31.5 | trace | 0.6 | — | 38.9 | 3.6 |

The proportion of DGLA, the immediate precursor of ARA, increased from about 1% in the wild type to over 30% in P127 under nitrogen starvation. It was thus assumed that the mutant was defective in its Δ5 desaturase gene. Interestingly, after 2 days (+N), the proportion of DGLA was only slightly lower than that of ARA in the WT. However, under N-starvation, the proportion of DGLA amounted to only one-half of that of ARA in the WT. Correspondingly, the share of oleic acid almost tripled. Instead of eicosapentaenoic acid (EPA, 20:5ω3) in the WT, the mutant produced eicosatetraenoic acid (ETA, 20:4ω3), indicating that the ω3(Δ17) desaturation of C20 PUFA was not affected. Also, the capacity of P127 to accumulate TAG was not impaired as indicated by the appearance of large oil bodies (not shown) and high TFA biomass content (see Table 1 above). The full-length sequence of the mutated Δ5 desaturase gene (MutPiDes5; SEQ ID No. 1) was obtained according to known methods by PCR amplification using the forward primer: CCAAAGCTTAAAATGATGGCTGTAACAGA and a reverse primer: GCTCTAGACTATCCCACGGTGGCCA, both primers containing the HindIII and XbaI restriction sites (underlined). The nucleotide sequence of the wild type gene was compared with that of the mutant using the CLUSTAL W2 program (http://www.ebi.ac.uk/Tools/clustalw2/index.html). The alignment showed that the 186^{th} nucleotide, downstream the start codon in the wild type gene, A (boldfaced), was replaced by the nucleotide G (boldfaced) in the MutPiDes5 gene (see FIG. 4).

To assure the absence of activity, the yeast pYMutPiDes5 transformants were fed with DGLA, the ω6 substrate of Δ5 desaturase, in the presence of Tergitol (1%) and Galactose (2%). DGLA was not desaturated by either the pYMutPiDes5 transformants or the empty pYES2-harboring negative control cells (not shown).

The expression profiles of VLC-PUFA biosynthesis genes [Δ12 (PiDes12), Δ6 (PiDes6), Δ5 (PiDes5) desaturases and Δ6 PUFA elongase (PiElo1) genes] in the WT and mutant P. incisa were compared by semi-quantative RT-PCR using actin, a constitutively expressed gene, as a control.

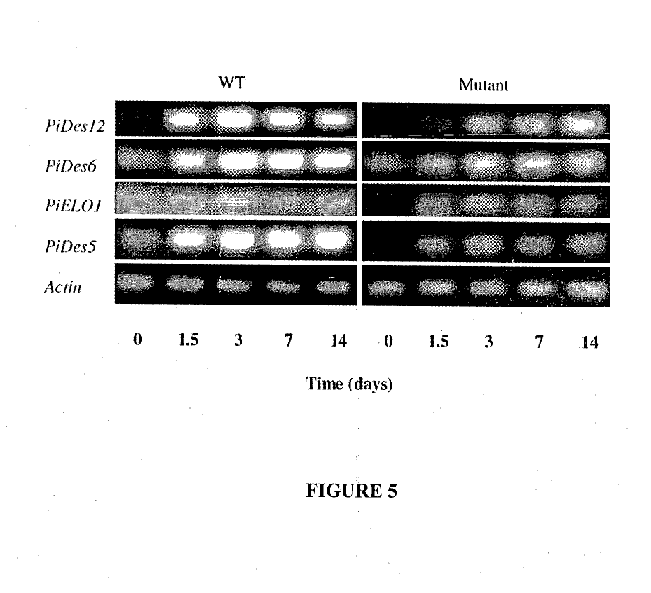

The results, displayed in FIG. 5, show that the transcript levels of all four genes appeared to be drastically reduced in the mutant. At the same time, the expression patterns of the Δ6 and Δ5 desaturase genes (PiDes6, PiDes5), as well as the Δ6 PUFA elongase gene (PiElo1), were not affected in the mutant, with the maximal transcript level being observed on the 3^{rd} day of N-starvation, however, at a substantially lower level compared to that of the wild type.

### 4) Use of the Mutant Carrying the MutPiDes5 Gene in Algal Transformation, for Example, in P. Incisa and Close Species

Plant transformation vectors, such as pCAMBIA, may be introduced into Parietochloris incisa mutant cells using biolistic delivery, electroporation or Agrobacterium-mediated technology. Mutant cells may be confirmed by sequencing the MutPiD5.DES gene as described above.

The pCAMBIA vector has a 35S promoter, a GUS reporter gene and a Hygromycin resistance gene which usually serves for selection of stable transformants. The WT PiD5DES gene will be introduced into the vector instead of the GUS reporter gene.

Following expression of the PiD5DES gene, the herbicide resistant colonies, whose growth on selective medium is confirmed in at least three subcultures, are analyzed by Gas Chromatography for the emergence of ARA. The appearance of significant levels of ARA is proof of successful transformation and development of the transformation protocol.

This methodology may be advantageously used to express genes conferring essential traits such as herbicide resistance, tolerance to high light intensity, tolerance to high salinity caused by water evaporation etc. Genetic modification of microalgae may be also used in metabolic engineering of algae to produce various nutritionally and pharmaceutically important PUFA, such as EPA and DHA.

1. An isolated nucleic acid molecule comprising the 186 by portion of the nucleotide sequence of SEQ ID NO. 1, said portion commencing at the start codon ATG.

2. The nucleic acid molecule according to claim 1 comprising SEQ ID NO. 1.

3. The nucleic acid molecule according to claim 1 wherein said nucleic acid molecule comprises cDNA or genomic DNA.

4. A vector comprising the isolated nucleic acid molecule of claim 1.

5. An isolated fresh water green algae cell comprising the nucleic acid molecule of claim 1.

6. The cell according to claim 5 wherein the alga is Parietochloris incisa.

7. A vector for algal transformation, the vector comprising a plant derived promoter, a WT PiD5DES gene and a gene to select for stable transformants.

8. The vector, according to. claim 7 wherein the plant derived promoter is 35S.

9. The vector according to claim 7 wherein the gene to select for stable transformants is a herbicide resistance gene.

10. A method for transformation of algae, the method comprising:
introducing into a MutPiDes5 mutant a vector according to claim 7;
selecting stable transformants; and
analyzing the FA composition of the MutPiDes5 mutant for the emergence of ARA.

11. The method according to claim 10 wherein selecting stable transformants comprises selecting herbicide resistant colonies.


SEQUENCE LISTING (1 sequence)

SEQ ID NO: 1 - DNA, 1446 bp, Parietochloris incisa
atgatggctgtaacagagggcgctgggggtgtaacggccgaggttggtttgcacaaacgcagttctcagccgcgtcccgcagctccccgcagcaagctgttcacgttggatgaggttgcaaagcacgacagcccgactgactgctgggtggtcattcggcggagggtttacgacgtgacggcgtgagtgccgcagcatcctggcggaaacctgatctttgtgaaagctggccgcgactgtacccagctgttcgattcctaccaccccttaagtgccagggctgtgctagacaagttctacatcggtgaagtcgatgtaaggcctggggacgagcagttccttgtggctttcgaagaggacacagaggagggtcagttctacacggtcctcaagaagcgtgtggagaagtacttcagggagaacaagctcaacccgcgggcaacaggcgccatgtacgccaagtcgctgaccatcctggcgggcctggcgttgagcttctatggtacgttctttgccttcagcagcgcaccggcctcgctgctcagcgctgtgctgctcggcatttgcatggcggaggtgggcgtgtccatcatgcacgatgccaaccacggcgcatttgcccgcaacacgtgggcctcgcatgccctgggcgccacgctggacatcgtgggggcatcctccttcatgtggcgccagcagcatgtcgtgggccaccatgcatacaccaacgtggacggtcaggacccagacctgcgagttaaggaccccgacgttcgccgcgtgaccaagttccagccccagcagtcgtaccaggcgtaccagcacatctacctggccttcctgtacggcctgctggccatcaagagcgtgctgctggacgactttatggccctcagctccggcgccatcggctccgtgaaagtggccaagctgacgcccggcgagaagctcgtgttctggggcggcaaggcgctctggctcggctactttgtgctgctgccggtggtgaagagccgccactcctggccgctgctggcggcctgctggctgctgagcgagtttgtcacgggctggatgctggccttcatgttccaggtggcgcacgtgaccagcgatgtgagctacctggaggctgacaagacaggcaaggtcccgaggggctgggctgccgcacaggccgccaccaccgccgactttgcgcatggctcctggttctggacccaaatttctggcggccttaactaccaggtggtgcaccatctgttcccgggcatctgccatctgcactacccggccatcgcccccatcgtgctggacacctgcaaggagtttaacgtgccctaccatgtgtaccccacgtttgtcagggcactcgccgcacacttcaagcatctcaaggacatgggcgccccaactgccatcccttcgctggccaccgtgggatag